(19) United States
(12) Patent Application Publication
(10) Pub. No.: US 2025/0230453 A1
(43) Pub. Date: Jul. 17, 2025

(54) ANTI-DISEASE GENE OF RICE AND USE THEREOF

(71) Applicant: Institute of Zoology, Chinese Academy of Sciences, Beijing (CN)

(72) Inventors: Feng CUI, Beijing (CN); Panpan GE, Beijing (CN); Hong LU, Beijing (CN); Yonghuan MA, Beijing (CN)

(73) Assignee: Institute of Zoology, Chinese Academy of Sciences, Beijing (CN)

(21) Appl. No.: 18/568,663

(22) PCT Filed: Mar. 16, 2023
(86) PCT No.: PCT/CN2023/081851

§ 371 Date: Dec. 8, 2023

(30) Foreign Application Priority Data
Sep. 19, 2022 (CN) 202211137305.8

(51) Int. Cl. C12N 15/82; C12N 15/10; C12N 15/11; C12Q 1/6806; C12Q 1/6895

(52) U.S. Cl. CPC C12N 15/8205; C12N 15/1096; C12N 15/11; C12N 15/8218; C12N 15/8283; C12Q 1/6806; C12Q 1/6895; C12N 2310/20; C12Q 2600/13

(57) ABSTRACT

The present application relates to the field of biotechnology, in particular to the use of Flotillin1 gene and Importin α4 gene and the use of molecules for inhibiting the transcription or translation of Flotillin1 gene and Importin α4 gene in the manufacture of kits. The present application also relates to a method for obtaining plants resistant to pathogens.

### TECHNICAL FIELD

The present application relates to the field of biotechnology, in particular to the use of rice Flotillin1 gene and Importin α4 gene, and the use of molecules inhibiting the transcription or translation of Flotillin1 gene and Importin α4 gene in the manufacture of a kit. The present application also relates to a method for obtaining a plant with resistance to a pathogen.

### BACKGROUND ART

Plant virus infection has become the second largest disease in agricultural production, and causes huge economic losses worldwide every year. Rice virus is one of the important groups of plant viruses, which seriously threatens the rice yield in East Asia, causing damages ranging from 16% yield reduction to total crop failure. Those with more serious outbreaks in recent years mainly include rice stripe virus (RSV), southern rice black-streaked dwarf virus (SRBSDV), rice black-streaked dwarf virus (RBSDV), rice ragged stunt virus (RRSV) and rice grassy stunt virus (RGSV). Therefore, blocking the spread of viruses in plant hosts and cultivating excellent varieties with broad-spectrum anti-virus performance are important ways to deal with viral diseases.

Rice stripe virus (RSV) is a single-stranded RNA virus, its genome comprises four RNA strands (RNA1, RNA2, RNA3, RNA4), which encode seven proteins (RdRp, NS2, NSvc2, NS3, NP, SP, NSvc4). At present, most of the researches on RSV and rice hosts focus on the interaction between proteins encoded by RSV and factors in the rice host, gene silencing, immunity, and autophagy pathways affecting virus's own replication or affecting plant host cells (Fu et al., 2018; Zheng et al., 2017; Zhao et al., 2016; Kong et al., 2014). However, the receptor of RSV in rice host remains unclear.

To sum up, plant virus infection seriously threatens global economic security and food safety, but research on blocking virus transmission is relatively scarce. There is a need to provide an anti-disease gene to deal with virus infection in rice.

### CONTENTS OF THE INVENTION

The present inventors of the present application have found through a lot of experiments and repeated explorations that Flotillin1 gene and Importin α4 in plants play a negative regulatory role on the resistance of plant against pathogens. Therefore, a rice mutant in which Flotillin1 and Importin α4 are knocked out is prepared, and it is unexpectedly found that the mutant significantly inhibits the spread rate of RSV in rice, and also significantly reduces the incidence of RSV in rice. Moreover, the mutant has no effect on rice plant height, thousand-grain weight and germination rate. Therefore, the mutant plants in which Flotillin1 gene and Importin α4 gene are knocked out have better application potential in plant disease resistance.

Therefore, in a first aspect, the present application provides a method for obtaining a plant capable of resisting a pathogen, the method comprising: reducing or inhibiting the transcription or translation of Flotillin1 gene and Importin α4 gene in the plant, thereby reducing or inhibiting the expression levels of the proteins encoded by the Flotillin1 gene and the Importin α4 gene in the plant.

In certain embodiments, the plant is a gramineous plant. In certain embodiments, the plant is selected from the group consisting of wheat, barley, corn, rice, sorghum.

In certain embodiments, the pathogen is a virus. In certain embodiments, the pathogen is a rice stripe virus.

In certain embodiments, the protein encoded by the Importin α4 gene has an amino acid sequence as is shown in SEQ ID NO: 3.

In certain embodiments, the protein encoded by the Flotillin1 gene has an amino acid sequence as shown in SEQ ID NO: 1.

In certain embodiments, reducing or inhibiting the expression of Flotillin1 gene and Importin α4 gene in the plant is carried out by any one of the following methods: substitution, deletion or addition of one or several nucleotides (e.g., substitution, deletion or addition of 1, 2 or 3 nucleotides), site-specific mutagenesis, ethyl methanesulfonate mutagenesis, directed induction of local mutation in genome, or gene editing (e.g., gene knockout guided by sgRNA).

In some embodiments, by designing sgRNA targeting Flotillin1 gene and Importin α4 gene, Cas9 is guided to edit the target site.

In certain embodiments, when several (e.g., 1, 2, 3, 4) copies of the Flotillin1 gene and the Importin α4 gene are present in the plant cell, each copy of the Flotillin1 gene and the Importin α4 gene becomes defective.

In a specific embodiment of the present invention, sgRNA sequences for rice Flotillin1 gene and Importin α4 gene are designed, respectively, and corresponding vectors containing sgRNA and cas9 proteins are constructed. Agrobacterium is used as a gene manipulation tool to carry the vectors to efficiently introduce the sgRNA sequences targeting the rice Flotillin1 gene and Importin α4 gene into the rice cell genome, reducing the expression levels of the rice Flotillin1 gene and Importin α4 gene, and significantly down-regulating the expression of the Flotillin1 gene and Importin α4 at mRNA level and protein level.

Accordingly, in certain embodiments, the method is accomplished by the following steps (a) to (f):
(a) constructing a vector containing sgRNA1 and sgRNA2, wherein the sgRNA1 is capable of targeting the Flotillin1 gene or fragment thereof, and the sgRNA2 is capable of targeting the Importin α4 gene or fragment thereof;
(b) transforming the vector into Agrobacterium;
(c) infecting plant cells with the Agrobacterium;
(d) optionally, selecting plant cells having defective Flotillin1 gene and Importin α4 gene;
(e) producing plants from the plant cells of step (c) or (d);
(f) optionally, screening a plant with defective Flotillin 1 gene and Importin α4 gene to obtain a plant resistant to a pathogen.

In certain embodiments, the sgRNA1 and sgRNA2 are constructed in the same vector or in different vectors. In certain embodiments, the sgRNA1 and sgRNA2 are constructed in pYLCRISPR/Cas9Pμbi-H vector.

In certain embodiments, the Agrobacterium is Agrobacterium EHA105.

In some embodiments, in step (f), RNA of the plant is extracted, the RNA is reverse transcribed into cDNA, primers are used to amplify nucleotide fragments targeted by the sgRNA1 and sgRNA2 in the cDNA for screening; or, genomic DNA of the plant is extracted, and primers are used to amplify nucleotide fragments targeted by the sgRNA1 and sgRNA2 in the DNA for screening.

In certain embodiments, the sgRNA1 has a nucleotide sequence as shown in SEQ ID NO: 5 or SEQ ID NO: 6.

In certain embodiments, the sgRNA2 has a nucleotide sequence as shown in SEQ ID NO: 7 or SEQ ID NO: 8.

In a second aspect, the present application provides a use of a molecule that specifically inhibits the transcription or translation of Flotillin1 gene and Importin α4 gene or specifically inhibits the expression levels of proteins encoded by Flotillin1 gene and Importin α4 gene in the manufacture of a kit, in which the kit is used to obtain a plant capable of resisting a pathogen, or to improve the ability of a plant to resist a pathogen.

In one embodiment of the present invention, the molecule includes but is not limited to nucleic acid molecule, carbohydrate, lipid, small molecule chemical drug, antibody drug, polypeptide, protein or interfering lentivirus.

In one embodiment of the present invention, the nucleic acid molecule includes but is not limited to: antisense oligonucleotide, double-stranded RNA (dsRNA), small interfering RNA (siRNA), short hairpin RNA (shRNA), or guide RNA (sgRNA).

In certain embodiments, the nucleic acid molecule comprises at least 2, at least 3, at least 4, at least 5, at least 6 sgRNAs.

In some embodiments, the nucleic acid molecule comprises sgRNA1 and sgRNA2, the sgRNA1 is capable of targeting the Flotillin1 gene or fragment thereof, and the sgRNA2 is capable of targeting the Importin α4 gene or fragment thereof.

In certain embodiments, the sgRNA1 has a nucleotide sequence as shown in SEQ ID NO: 5 or SEQ ID NO: 6.

In certain embodiments, the sgRNA2 has a nucleotide sequence as shown in SEQ ID NO: 7 or SEQ ID NO: 8.

In some embodiments, the kit further comprises: a vector, Agrobacterium, a medium and/or reagent for culturing a plant cell or tissue, a reagent for extracting plant DNA or RNA, primers for amplifying fragments separately targeted by sgRNA1 and sgRNA2, or any combination thereof.

In certain embodiments, the vector is pYLCRISPR/Cas9Pμbi-H.

In certain embodiments, the sgRNA1 and sgRNA2 are contained or not contained in the vector.

In certain embodiments, the Agrobacterium is Agrobacterium EHA105.

In certain embodiments, the plant is a gramineous plant. In certain embodiments, the plant is selected from the group consisting of wheat, barley, corn, rice, sorghum.

In certain embodiments, the pathogen is a virus. In certain embodiments, the pathogen is a rice stripe virus.

In certain embodiments, the protein encoded by Importin α4 gene has an amino acid sequence as shown in SEQ ID NO: 3.

In certain embodiments, the protein encoded by Flotillin1 gene has an amino acid sequence as shown in SEQ ID NO: 1.

In a third aspect, the present application provides a kit, comprising any one or more of the following items (1) to (6):
(1) a nucleic acid molecule that specifically inhibits the transcription or translation of Flotillin1 gene and Importin α4 gene, or specifically inhibits the expression levels of proteins encoded by Flotillin1 gene and Importin α4 gene;
(2) a vector comprising the nucleic acid molecule;
(3) an Agrobacterium containing the vector;
(4) a medium and/or reagent for culturing a plant cell or tissue;
(5) a reagent for extracting plant DNA or RNA;
(6) a primer set for amplifying a nucleotide fragment comprising Flotillin 1 gene and Importin α4 gene.

In certain embodiments, the kit comprises all combinations of (1) to (6).

In certain embodiments, the nucleic acid molecule is selected from the group consisting of antisense oligonucleotide, dsRNA, siRNA, shRNA, or sgRNA.

In some embodiments, the nucleic acid molecule comprises sgRNA1 and sgRNA2, the sgRNA1 is capable of targeting the Flotillin1 gene or fragment thereof, and the sgRNA2 is capable of targeting the Importin α4 gene or fragment thereof.

In some embodiments, wherein the kit is used to obtain a plant capable of resisting a pathogen, or to improve the ability of a plant to resist a pathogen.

In certain embodiments, the plant is a gramineous plant. In certain embodiments, the plant is selected from the group consisting of wheat, barley, corn, rice, sorghum.

In certain embodiments, the pathogen is a virus. In certain embodiments, the pathogen is a rice stripe virus.

In certain embodiments, the protein encoded by the Importin α4 gene has an amino acid sequence as shown in SEQ ID NO: 3.

In certain embodiments, the protein encoded by the Flotillin1 gene has an amino acid sequence as shown in SEQ ID NO: 1.

In certain embodiments, the sgRNA1 has a nucleotide sequence as shown in SEQ ID NO: 5 or SEQ ID NO: 6.

In certain embodiments, the sgRNA2 has a nucleotide sequence as shown in SEQ ID NO: 7 or SEQ ID NO: 8.

In certain embodiments, the vector is pYLCRISPR/Cas9Pμbi-H.

In certain embodiments, the Agrobacterium is Agrobacterium EHA105.

### Definition of Terms

In the present invention, unless otherwise specified, the scientific and technical terms used herein have the meanings commonly understood by those skilled in the art. Moreover, the operational steps of molecular genetics, nucleic acid chemistry, chemistry, molecular biology, biochemistry, cell culture, microbiology, cell biology, genomics and recombinant DNA used herein are all routine procedures widely used in the corresponding fields. Meanwhile, in order to better understand the present invention, definitions and explanations of related terms are provided below.

As used herein, the term “defective” refers to a reduction in the level of protein encoded by a gene in a cell or plant as compared to a wild-type plant, and the reduction can be about 10%, 20%, 30%, 40%, 50%, 60%, 70%, 80%, 90%, or 100% (i.e., the level at which the protein is completely inhibited).

As used herein, the term “protein encoded by Flotillin1 gene” refers to a naturally occurring, biologically active Flotillin1 protein. The Flotillin1 protein exists in a variety of gramineous plants, and the amino acid sequence of the protein can be easily obtained from various public databases (e.g., GenBank database). In certain embodiments, the amino acid sequence of the protein encoded by the Flotillin1 gene is shown in SEQ ID NO: 1.

As used herein, the term “protein encoded by Importin α4 gene” refers to a naturally occurring, biologically active Importin α4 protein. The Importin α4 protein is present in a variety of gramineous plants, and the amino acid of the protein can be readily obtained from various public databases (e.g., GenBank database). In certain embodiments, the amino acid sequence of the protein encoded by the Importin α4 gene is shown in SEQ ID NO: 3.

As used herein, the term “Flotillin1 gene” refers to any nucleic acid encoding Flotillin1 protein, including DNA (e.g., genomic DNA), RNA (e.g., mRNA). In certain embodiments, the nucleotide sequence of Flotillin1 gene is shown in SEQ ID NO: 2.

As used herein, the term “Importin α4 gene” refers to any nucleic acid encoding Importin α4 protein, including DNA (e.g., genomic DNA), RNA (e.g., mRNA). In certain embodiments, the nucleotide sequence of Importin α4 gene is shown in SEQ ID NO: 4.

As used herein, the term “pathogen” refers to all organisms capable of causing a disease in a plant, including fungi, nematodes, bacteria and viruses. In certain embodiments, the pathogen is a rice stripe virus.

As used herein, the term “plant capable of resisting pathogen” refers to a plant that can resist the infection and/or spread of the pathogens to a certain extent, specifically be manifested in that after the pathogen infects the plant, as compared to a wild-type plant, the plant has an alleviated morbidity.

As known to those skilled in the art, codon degeneracy exists. That is, in the process of protein translation, each amino acid may correspond to one or more codons, for example, up to 6 codons. Different species have great differences in the use of degenerate codons to encode a certain amino acid, and have different biases. This bias phenomenon is called “codon bias”. Therefore, as used herein, the term “codon bias” refers to the situation where a species prefers to use certain specific codons to encode amino acids. Optimizing the sequence of a nucleic acid molecule according to codon bias is particularly advantageous in certain circumstances, for example, possibly helping to increase the expression level of a protein encoded by the nucleic acid molecule.

As used herein, the term “vector” refers to a nucleic acid delivery vehicle into which a polynucleotide can be inserted. When the vector is capable of achieving the expression of the protein encoded by the inserted polynucleotide, the vector is called an expression vector. A vector can be introduced into a host cell by transformation, transduction or transfection, so that the genetic material elements it carries can be expressed in the host cell. Vectors are well known to those skilled in the art, including but not limited to: plasmids (e.g., naked plasmids); phagemids; cosmids; artificial chromosomes, such as yeast artificial chromosome (YAC), bacterial artificial chromosome (BAC) or P1-derived artificial chromosome (PAC); bacteriophage such as λ-phage or M13 phage and viral vectors, etc. Viruses that can be used as vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40). A vector may contain a variety of elements that control expression, including but not limited to, promoter sequence, transcription initiation sequence, enhancer sequence, selection element, and reporter gene. In addition, the vector may also contain an origin of replication.

As used herein, the term “sgRNA (small guide RNA)” is a guide RNA used to target a target nucleic acid, and under the guidance of the sgRNA, the Cas9 protein performs site-specific editing of the target nucleic acid. Usually, in the CRISPR/Cas9 genome editing technology, the sgRNA targeting the target nucleic acid is designed to guide the Cas9 nuclease to target a specific site of the target nucleic acid. Not every sgRNA has equal cutting efficiency. In view of this inconsistency, it is necessary to screen multiple sgRNAs to find the one with the highest cutting efficiency. In certain embodiments, the sgRNA targeting Flotillin1 gene has a nucleotide sequence as shown in SEQ ID NO: 5 or SEQ ID NO: 6. In certain embodiments, the sgRNA targeting Importin α4 gene has a nucleotide sequence as shown in SEQ ID NO: 7 or SEQ ID NO: 8.

### Beneficial Effects of the Invention

The applicant prepared rice mutants with single-knockout of Importin α4 or Flotillin1, respectively, and found that the effect of the single-knockout mutants were not ideal through experimental tests; in addition, the applicant unexpectedly found that rice mutant with knockout of both Flotillin1 and Importin α4 can significantly suppress the spread rate of RSV in rice (suppressing at least 50% the spread rate as compared with WT), and can significantly reduce the incidence of RSV in rice (reducing at least 40% the incidence as compared with WT). Moreover, the mutant has no effect on rice plant height, thousand-grain weight and germination rate. Therefore, the mutant and its knockout combination (i.e., knockout of Flotillin1 gene and Importin α4 gene) have good application potentials in plant disease resistance.

Embodiments of the present invention will be described in detail below with reference to the drawings and examples, but those skilled in the art will understand that the following drawings and examples are only for illustrating the present invention, rather than limiting the scope of the present invention. Various objects and advantages of the present invention will become apparent to those skilled in the art from the accompanying drawings and the following detailed description of the preferred embodiments.

### BRIEF DESCRIPTION OF THE DRAWINGS

FIG. 1 shows the verification results of transcript level and protein level of rice Flotillin1 mutant, wherein FIG. 1 A shows the expression levels for Flotillin1 transcript level in wild-type rice (WT) and Flotillin1 mutant; FIG. 1 B shows the expression levels for Flotillin1 protein level in wild-type rice (WT) and Flotillin1 mutant. The values are expressed as mean±standard error, and different lowercase letters indicate significant differences in gene expression among different treatments (P<0.05).

FIG. 2 shows the effect of knocking out Flotillin1 on rice plant height, thousand-grain weight and germination rate, wherein, FIG. 2 A and B represent changes in rice plant height after knocking out Flotillin1, and the scale is 10 cm; FIG. 2 C and D represent the change of thousand-grain weight of rice after knocking out Flotillin1, and the scale bar is 5 cm. The values are expressed as mean±standard error, and different lowercase letters indicate significant differences in gene expression among different treatments (P<0.05).

FIG. 3 shows the effect of knocking out Flotillin1 on the spread rate of RSV in rice, wherein, FIG. 3 A and B represent spread rates in epidermal cells, fiber tissues, mesophyll cells, bundle sheath cells, sieve tubes and companion cells after RSV infects the inoculated leaves for 12 hours in Flotillin1 mutant, the scale bar is 20 nm; FIG. 3 C and D represent spread rates in epidermal cells, fiber tissues, mesophyll cells, bundle sheath cells, sieve tubes and companion cells after RSV infects the systemic leaves for 1 day in Flotillin1 mutant, the scale bar is 20 nm.

Epi represents epidermal cells, Fib represents fibrous tissue, Mes represents mesophyll cells, Bs represents bundle sheath cells, SE represents sieve tubes, and CC represents companion cells.

Blue fluorescence indicates the signal of plasmodesmata staining, and red fluorescence indicates the signal of RSV NP. The values are expressed as mean±standard error, and different lowercase letters indicate significant differences in gene expression among different treatments (P<0.05).

FIG. 4 shows the effect of knocking out Flotillin1 on the expression of RSV NP in rice, wherein FIG. 4 A, B and C show the change of NP expression levels after RSV infects rice systemic leaves for 1d, 4d, and 7d, respectively, in the Flotillin1 mutant. The values are expressed as mean±standard error, and different lowercase letters indicate significant differences in gene expression among different treatments (P<0.05).

FIG. 5 shows the effect of knocking out Flotillin1 on rice disease incidence, wherein, FIG. 5 A shows the effect of RSV infection on rice disease incidence in Flotillin1 mutant when the insect population density is 10 heads, and FIG. 5 B shows the effect of RSV infection on rice disease incidence of Flotillin1 mutant when the insect population density is 2 heads. The values are represented by mean±standard error, asterisks indicate significant differences among treatments, *P<0.05, **P<0.01, and different lowercase letters indicate significant differences in gene expression among different treatments (P<0.05).

FIG. 6 shows the effect of knocking out Importin α4 on rice plant height and thousand-grain weight, wherein FIG. 6 A and B show the change in rice plant height after knocking out Importin α4, and the scale is 10 cm; FIG. 6 C shows the change in rice thousand-grain weight after knocking out Importin α4. The values are expressed as mean±standard error, and different lowercase letters indicate significant differences in gene expression among different treatments (P<0.05).

FIG. 7 shows the effect of knocking out Importin α4 on expression level of RSV NP in rice, wherein FIG. 7 A, B and C respectively show the change in NP expression level after RSV infects rice systemic leaves for 1d, 4d and 7d in Importin α4 mutant. The values are expressed as mean standard error, and different lowercase letters indicate significant differences in gene expression among different treatments (P<0.05).

FIG. 8 shows the effect of knocking out Importin α4 on rice disease incidence. FIG. 8 A shows the effect of RSV infection on rice disease incidence in Importin α4 mutant when the population density is 10 heads. FIG. 8 B shows the effect of RSV infection on rice incidence in Importin α4 mutant when the population density is 2 heads. The values are expressed as mean±standard error, asterisks indicate significant differences among treatments, *P<0.05, **P<0.01, different lowercase letters indicate significant differences in gene expression among different treatments (P<0.05).

FIG. 9 shows the effect of knocking out Flotillin1-Importin α4 on rice plant height and thousand-grain weight, wherein FIG. 9 A and B show changes in rice plant height after knocking out Flotillin1-Importin α4, and the scale bar is 10 cm; FIG. 9 C shows changes in rice thousand-grain weight after knocking out Flotillin1-Importin α4. The values are expressed as mean standard error, and different lowercase letters indicate significant differences in gene expression among different treatments (P<0.05).

FIG. 10 shows the effect of knocking out Flotillin1-Importin α4 on expression level of RSV NP in rice, wherein FIG. 10 A, B and C show changes in NP expression level after RSV infects rice systemic leaves for 1d, 4d and 7d in Flotillin1-Importin α4 mutant. The values are expressed as mean±standard error, and different lowercase letters indicate significant differences in gene expression among different treatments (P<0.05).

FIG. 11 shows the effect of knocking out Flotillin1-Importin α4 on rice disease incidence. FIG. 11 A shows the effect of RSV infection on rice disease incidence in Flotillin1-Importin 4 mutant when the population density is 10 heads. FIG. 11 B shows the effect of RSV infection on rice disease incidence in Flotillin1-Importin α4 mutant when the population density is 2 heads. The values are represented by mean±standard error, asterisks indicate significant differences among treatments, *P<0.05, **P<0.01, different lowercase letters indicate significant differences in gene expression among different treatments (P<0.05).

### SEQUENCE INFORMATION

Information on the partial sequences involved in the present invention is provided in Table 1 below.

TABLE 1
Description of the sequences

| SEQ ID | | |
|---|---|---|
| NO: | Description | Sequence |
| 1 | Amino acid | MGFAYRIASASEYLAITGYGIADVKLAKKAWVAPGQRCTRFDI |
| | sequence of | SPVNYTFEVQAMSAEKLPFILPAVFTIGPRADDDDCLLRYAKL |
| | Flotillin1 | ISPHDKLSHHVNELVKGVIEGETRVLAASMTMEEIFQGTKSFK |
| | | QAVFENVQLELNQFGLIIYNANVKQLVDVAGHEYFSYLGQKTQ |
| | | QEAVNQAKVDVAEARMKGEVGAKERDGMTRQNAAKVDAETKVY |
| | | TVKRQGEGAKEEARVKAEVKVFENEREAEVAEANADLAMKKAG |
| | | WQRQAMVAEVEAAKAVAIREAELQVEVERTNASRQTEKLKAEH |
| | | LSKAVVDYEMKVQEANWELYNRQKAAEALLYEQEKQAEARRAS |
| | | ADAAFFARQREAEAELYAKQKEAEGLVAMGDAQSAYLSAMLGA |
| | | LGGSYAALRDYLMVSSGVYQEMARINADAIRGLEPKISVWSNG |
| | | AGAGGEVGEGGGAMKEVAGVYKMLPPLLTTVHEQTGMLPPAWM |
| | | GTLTGGAPSSTS |
| 2 | Nucleotide | ATGGGGTTCGCGTACAGGATCGCGAGCGCGTCGGAGTACCTGG |
| | sequence of | CGATCACCGGGTACGGCATCGCCGACGTGAAGCTGGCGAAGAA |
| | Flotillin1 | GGCGTGGGTGGCGCCGGGGCAGCGGTGCACCCGCTTCGATATC |
| | | TCCCCGGTGAACTACACCTTCGAGGTGCAGGCCATGAGCGCCG |
| | | AGAAGCTCCCCTTCATCCTCCCGGCCGTCTTCACCATCGGCCC |
| | | CCGCGCCGACGACGACGACTGCCTCCTCCGCTACGCCAAGCTC |
| | | ATCTCCCCGCACGACAAGCTCTCCCACCACGTCAACGAGCTCG |
| | | TCAAGGGCGTCATCGAGGGTGAGACCCGCGTGCTGGCCGCCTC |
| | | CATGACCATGGAGGAGATCTTCCAGGGCACCAAGTCCTTCAAG |
| | | CAGGCCGTCTTCGAGAACGTCCAGCTGGAGCTCAACCAGTTCG |
| | | GCCTCATCATCTACAACGCCAACGTCAAGCAGCTCGTCGACGT |
| | | CGCCGGCCACGAGTACTTCTCCTACCTCGGCCAGAAGACGCAG |
| | | CAGGAGGCGGTGAACCAGGCGAAGGTGGACGTCGCGGAGGCGC |
| | | GGATGAAGGGGGAGGTCGGCGCCAAGGAGAGGGACGGGATGAC |
| | | GCGGCAGAACGCCGCCAAGGTGGACGCCGAGACGAAGGTGTAC |
| | | ACGGTGAAGCGGCAGGGCGAGGGGGCGAAGGAGGAGGCGAGGG |
| | | TGAAGGCGGAGGTGAAGGTGTTCGAGAACGAGAGGGAGGCGGA |
| | | GGTGGCCGAAGCGAACGCTGACCTGGCGATGAAGAAGGCCGGG |
| | | TGGCAACGACAGGCGATGGTGGCTGAAGTGGAGGCCGCCAAGG |
| | | CGGTCGCCATTCGTGAAGCCGAGCTGCAGGTGGAGGTGGAACG |
| | | GACTAACGCCTCTAGGCAGACTGAGAAGCTCAAGGCCGAGCAT |
| | | CTCAGCAAGGCTGTCGTCGACTACGAGATGAAGGTGCAAGAAG |
| | | CGAACTGGGAGCTGTACAACCGGCAGAAGGCGGCGGAGGCTCT |
| | | GCTGTACGAGCAGGAGAAGCAGGCGGAGGCGCGGCGCGCGTCG |
| | | GCGGACGCGGCCTTCTTCGCGCGGCAGCGCGAGGCCGAGGCGG |
| | | AGCTCTACGCCAAGCAGAAGGAGGCCGAGGGCCTGGTGGCCAT |
| | | GGGCGACGCCCAGAGCGCCTACCTCTCCGCCATGCTCGGCGCG |
| | | CTCGGCGGCAGCTACGCCGCGCTCCGGGACTACCTCATGGTCA |
| | | GCTCCGGCGTGTACCAGGAGATGGCGCGCATCAACGCCGACGC |
| | | CATCAGGGGGCTGGAGCCCAAGATCAGCGTGTGGAGCAACGGC |
| | | GCCGGCGCCGGCGGCGAGGTCGGCGAAGGCGGTGGCGCGATGA |
| | | AGGAGGTGGCCGGGGTGTACAAGATGCTGCCGCCGCTGCTGAC |
| | | GACGGTGCACGAGCAGACCGGGATGCTGCCGCCGGCGTGGATG |
| | | GGCACTCTGACTGGCGGCGCCCCCTCGTCGACCAGTTGA |
| 3 | Amino acid | MVEKVWSDDTTSQLEATIQFRRLLSDEKNPTVIKIIRADVLPR |
| | sequence of | FSDFLSRHEHPQLQMEAAWVLTNIAASDYTLLVAECGAVPRLV |
| | Importin α4 | ELLESANANIRHQAIWALGNIAADVPTCREIVLDHGAVTPLLA |
| | | QFREGMKVPVLRTATWALSNLCFGKLPAEVQVKPILDIISQLI |
| | | HSVDEKILGDACWALCYICDGVSDGIQHVLDAGACPQLVNLLM |
| | | HASANILLPVITVLARISSGDDAQVQVLVENDILNYLAPLLAR |
| | | NYPKSIKKQAYLIVSNISTGSKDNIQAVIDADVISPLIFLLKT |
| | | SEKDIKEEAAWAISNAASGGSNDQIQYLVSRRCLEPLCNVLTY |
| | | QDADLVYACLEGLQNILQAGAVGKQGQGSTVNPYAQFILECGG |
| | | LDKLEDLQEVDNDAIYKLVMKLLEGYWDEEVSDDDPNLPTSND |
| | | SAETVETASEDAAQPTEPSASPNESE |
| 4 | Nucleotide | ATGGTGGAGAAGGTCTGGTCAGATGATACCACTAGTCAGTTAG |
| | sequence of | AAGCCACAATCCAATTCAGGAGACTCCTTTCAGATGAGAAGAA |
| | Importin α4 | CCCAACCGTGATAAAAATCATCAGAGCAGATGTCCTGCCAAGG |
| | | TTTTCTGATTTCCTCTCAAGACATGAGCATCCTCAGCTACAAA |
| | | TGGAGGCGGCATGGGTTCTTACCAACATAGCTGCATCTGACTA |
| | | TACATTGCTAGTTGCAGAATGTGGTGCTGTTCCAAGGTTGGTC |
| | | GAGCTCTTAGAATCTGCAAATGCTAATATCAGGCATCAGGCTA |
| | | TCTGGGCTCTTGGAAATATAGCTGCAGACGTGCCTACCTGCAG |
| | | AGAGATCGTCCTTGATCATGGTGCTGTGACACCATTACTTGCC |
| | | CAATTTAGAGAAGGCATGAAAGTTCCAGTTCTGAGGACTGCCA |
| | | CTTGGGCGTTGTCAAACCTTTGTTTTGGAAAATTACCAGCTGA |
| | | AGTGCAAGTGAAGCCAATACTGGATATAATCAGCCAGCTTATT |
| | | CATTCAGTCGACGAAAAGATACTGGGTGATGCATGCTGGGCTC |
| | | TTTGTTATATATGTGACGGTGTATCTGACGGAATTCAACATGT |
| | | GCTAGATGCGGGTGCTTGCCCTCAACTTGTAAATCTTTTGATG |
| | | CATGCATCAGCTAATATCCTGCTTCCTGTCATTACGGTACTTG |
| | | CGAGAATCTCTTCTGGAGATGATGCTCAAGTGCAGGTCCTAGT |
| | | AGAAAATGACATTCTTAATTATTTGGCCCCGTTGCTAGCACGA |
| | | AATTACCCAAAGAGCATCAAGAAGCAAGCTTATCTAATTGTTT |
| | | CTAATATCTCCACTGGCAGCAAGGATAATATTCAGGCAGTAAT |
| | | TGATGCAGATGTTATAAGCCCCCTCATTTTCCTATTAAAGACC |
| | | TCCGAGAAGGATATCAAGGAGGAAGCTGCTTGGGCTATATCAA |
| | | ATGCTGCGTCTGGTGGTTCAAATGATCAAATTCAATATTTGGT |
| | | GAGTCGAAGATGTTTGGAGCCACTCTGCAATGTTCTCACTTAC |
| | | CAAGATGCTGACCTAGTATATGCGTGCCTGGAAGGTCTTCAAA |
| | | ACATACTTCAGGCGGGTGCAGTGGGGAAGCAGGGGCAGGGTTC |
| | | CACGGTGAACCCGTATGCACAGTTTATTTTAGAATGTGGGGGT |
| | | TTGGATAAACTGGAAGATCTGCAGGAAGTCGACAATGATGCCA |
| | | TTTACAAGTTAGTCATGAAGTTGCTGGAGGGTTACTGGGACGA |
| | | GGAAGTAAGTGATGATGACCCAAATTTACCAACTTCAAATGAC |
| | | TCTGCAGAGACCGTGGAGACGGCATCTGAAGATGCTGCGCAGC |
| | | CAACAGAGCCATCGGCCAGCCCAAATGAAAGCGAATGA |
| 5 | SgRNA-1 | GTGGGAGAGCTTGTCGTGCGGGG |
| 6 | SgRNA-2 | CTCGAAGACGGCCTGCTTGAAGG |
| 7 | SgRNA-3 | TCAGAGCAGATGTCCTGCCAAGG |
| 8 | SgRNA-4 | TCTAAGAGCTCGACCAACCTTGG |
| 9 | Q-OsFlot1-F | GAACTACACCTTCGAGGTGC |
| 10 | Q-OsFlot1-R | TGCGTCTTCTGGCCGAGGT |
| 11 | Q-OsIMPα4-F | TGTGGTGCTGTTCCAAGGTT |
| 12 | Q-OsIMPα4-R | GGCTTCACTTGCACTTCAGC |
| 13 | RSV NP-F | GGAACAAATGCCAATGCTATC |
| 14 | RSV NP-R | TGAGACATTTGGGAATAGCTGA |
| 15 | UBQ10-F | TGGTCAGTAATCAGCCAGTTTGG |
| 16 | UBQ10-R | GCACCACAAATACTTGACGAACAG |
| 17 | Forward | GAACTACACCTTCGAGGTGC |
| | primer F1:5 | |
| 18 | Reverse | TGCGTCTTCTGGCCGAGGT |
| | primer R1:5 | |
| 19 | Forward | TGTGGTGCTGTTCCAAGGTT |
| | primer F1:6 | |
| 20 | Reverse | GGCTTCACTTGCACTTCAGC |
| | primer R1:6 | |

### Specific Models for Carrying Out the Invention

The present invention will now be described with reference to the following examples, which are intended to illustrate the present invention, not limit it.

Unless otherwise indicated, the experiments and methods described in the examples were essentially performed according to conventional methods well known in the art and described in various references. For example, the conventional techniques of immunology, biochemistry, chemistry, molecular biology, microbiology, cell biology, genomics and recombinant DNA used in the present invention can be found in Sambrook, Fritsch and Maniatis, MOLECULAR CLONING: A LABORATORY MANUAL, 2nd ed. (1989); CURRENT PROTOCOLS IN MOLECULAR BIOLOGY, edited by F. M. Ausubel et al. (1987); METHODS IN ENZYMOLOGY, series, Academic Publishing Corporation: PCR 2: A PRACTICAL APPROACH, edited by M. J. MacPherson, B. D. Hamres, and G. R. Taylor (1995); and ANIMAL CELL CULTURE, edited by R. I. Freshney (1987).

In addition, those without giving specific conditions in the examples were carried out according to conventional conditions or conditions recommended by the manufacturers. The reagents or instruments used without giving manufacturers were all commercially available conventional products. Those skilled in the art understand that the examples describe the present invention by way of example and are not intended to limit the scope of the present invention. All publications and other references mentioned herein are incorporated by reference in their entirety.

### Example 1: Insects to be Tested and Plants to be Tested

Insects to be tested: The virus-carrying and virus-free strains of SBPH (Laodelphax striatellus) used herein were mainly collected from Hai'an, Jiangsu Province and domesticated for a long time. The SBPHs were reared in glass jars containing 2-3 cm rice seedlings and sealed with nylon mesh. The rearing temperature was maintained at about 25° C., and the photoperiod was 16 h:8 h (light:dark). In order to ensure sufficient nutrition, the rice seedlings were replaced once a week. The virus-carrying strain and virus-free strain of SBPH were reared in different greenhouses with the same environmental conditions, and the virus loads were detected and screened by dot enzyme-linked immunosorbent immunoassay every three months.

Screening of virus-carrying SBPH: In order to obtain a strain of SBPH with high infection rate, we regularly screened the infected population. 40 to 50 female SBPHs to be born were taken and reared in separate bottles. When the larvae to be produced were hatched, 5 larvae were randomly selected from each bottle, and the virus loads of the larvae of was detected by the method of dot enzyme-linked immunosorbent immunoassay. Among the detected larvae, all female virus-carrying SBPH progenies were mixed and reproduced, and used as a population of virus-carrying stain line. The specific operation steps of dot enzyme-linked immunosorbent immunoassay were as follows:
(1) SBPHs to be tested were placed in 0.2 mL PCR tubes, one for each tube, added with 5 μL of 0.05M carbonate buffer, and ground with a top-treated tip.
(2) 3 μL of the ground sample was taken and placed on nitrocellulose membrane, then placed in an incubation box, and air-dried in a fume hood.
(3) Blocking: 10 mL of 1% skimmed milk (prepared with 1× PBST buffer solution) was added to the incubation box containing the dried nitrocellulose membrane, placed on a shaker, and blocked at room temperature for 30 min.
(4) Incubation of primary antibody: After the blocking solution was poured off, RSV NP monoclonal antibody (Zhao, et al., 2016) was diluted with 1% skimmed milk at a ratio of 1:5000, and 10 mL thereof was added to the incubation box, placed on a shaker, and incubated at room temperature for 2 h.
(5) Washing primary antibody: After the primary antibody diluent was poured off, lx PBST buffer was added for washing on a shaker for 15 min, and the washing was repeated 2-3 times.
(6) Incubation of secondary antibody: Goat anti-mouse secondary antibody linked with horseradish peroxidase (HRP) (Kangwei Century, CW0102S) was diluted with 1% skimmed milk at a ratio of 1:10000, and 10 mL thereof was added to the incubation box, placed on a shaker, and incubated at room temperature for 1 h.
(7) Washing secondary antibody: After the secondary antibody diluent was poured off, lx PBST buffer was added for washing on a shaker for 15 min, and the washing was repeated 2-3 times.
(8) The cleaned membrane was placed in a chromogenic solution (10 mL of 2 mM PBS solution, 2 mL of absolute ethanol, 6 μg of 4-chloro-1-naphthol, and 7 μL of 30% H₂O₂), and color development was carried out on a shaker at room temperature for 2 h.
(9) After color development, it was rinsed with tap water three times and air-dried at room temperature. The appearance of blue-purple spots indicated that the sample carried virus.

Plants to be tested: Nipponbare rice (Oryza sativa Japonica Gropp) was cultivated in an artificial culture rack at a temperature of 26° C./24° C., a photoperiod of L:D=14:10, and a relative air humidity of 70%, and used for experiments after 45 days of culture.

### Example 2: Preparation of Rice Mutants

### 1. Rice Flotillin1 Mutant

(1) Vector construction: Two targeting sgRNAs (Table 2) of Flotillin1 (its amino acid sequence was shown in SEQ ID NO: 1, and its nucleotide sequence was shown in SEQ ID NO: 2) were designed, and sequentially connected into CRISPR/Cas9 binary vector pYLCRISPR/Cas9Pμbi-H (Wuhan Boyuan Biotech).

TABLE 2
Sequences of sgRNAs of Flotillin1

| Gene Name | Primer Sequence (5′-3′) |
|---|---|
| SgRNA-1 | GTGGGAGAGCTTGTCGTGCGGGG |
| SgRNA-2 | CTCGAAGACGGCCTGCTTGAAGG |

(2) The target vector was used to transform Agrobacterium EHA105, and transferred into Nipponbare rice embryogenic calli by Agrobacterium mediation method. For the Flotillin1 gene, more than 3 homozygous mutants in which Flotillin1 gene was knockout were identified.
(3) Molecular identification of Flotillin1 mutant rice: The transgenic plant DNA was extracted, primers at both ends of the targeting sgRNAs were designed, PCR was carried out to expand the fragments containing targeting region, and sequencing was carried out to analyze the targeting effect. The sequences of primers F1 and R1 for targeting effect detection were as follows: forward primer F1:5 (nucleotide sequence shown in SEQ ID NO: 15), reverse primer R1:5 (nucleotide sequence shown in SEQ ID NO: 16).

### 2. Rice Importin α4 Mutant

(1) Vector construction: Two targeting sgRNAs (Table 3) of Importin α4 (its amino acid sequence was shown in SEQ ID NO: 3, and its nucleotide sequence was shown in SEQ ID NO: 4) were designed, and sequentially connected into CRISPR/Cas9 binary vector pYLCRISPR/Cas9Pμbi-H (Wuhan Boyuan Biotech).

TABLE 3
Sequences of sgRNAs of OsImportin α4

| Gene Name | Primer Sequences (5′-3′) |
|---|---|
| SgRNA-3 | TCAGAGCAGATGTCCTGCCAAGG |
| SgRNA-4 | TCTAAGAGCTCGACCAACCTTGG |

(2) The target vector was used to transform Agrobacterium EHA105, and transferred into Nipponbare rice embryogenic calli by Agrobacterium-mediation method. For the Importin α4 gene, more than 3 homozygous mutants in which Importin α4 gene was knocked out were identified.
(3) Molecular identification of Importin α4 mutant rice: The transgenic plant DNA was extracted, primers at both ends of the targeting sgRNAs were designed, PCR was carried out to expand the fragments containing the targeting region, and sequencing was carried out to analyze the targeting effect. The sequences of primers F1 and R1 for targeting effect detection were as follows: forward primer F1:6 (nucleotide sequence shown in SEQ ID NO: 19), reverse primer R1:6 (nucleotide sequence shown in SEQ ID NO: 20).

### 3. Rice Importin α4-Flotillin1 Mutant

(1) Vector construction: Two targeting sgRNAs of Flotillin1 (its amino acid sequence was shown in SEQ ID NO: 1, and its nucleotide sequence was shown in SEQ ID NO: 2) (its specific sequence was the same as the above Table 2) and two targeting sgRNAs of OsImportin α4 (its amino acid sequence was shown in SEQ ID NO: 3, and its nucleotide sequence was shown in SEQ ID NO: 4) (its specific sequence was the same as the above Table 3) were designed, and sequentially connected into CRISPR/Cas9 binary vector pYLCRISPR/Cas9Pμbi-H.
(2) The target vector was used to transform Agrobacterium EHA105, and transferred into Nipponbare rice by Agrobacterium-mediation method. For the Importin α4 gene and Flotillin1 gene, the homozygous mutant in which Importin α4 and Flotillin1 genes were knocked out was identified.
(3) Molecular identification of Importin α4 mutant rice: The transgenic plant DNA was extracted, primers at both ends of the targeting sgRNAs were designed, PCR was carried out to expand the fragments containing the targeting region, and sequencing was carried out to analyze the targeting effect.

### Example 3: Verification and Testing Steps of Rice Mutants

1. RNA Extraction from Rice, cDNA Synthesis and qPCR Quantification
(1) RNA extraction: RNA was extracted from rice leaves using the Trizol method (Ambion, 15596018), and the specific steps were as follows:

200 mg of rice leaves was taken and placed in a 1.5 mL centrifuge tube, added with 1 mL of Trizol reagent (Ambion, 15596018) to grind the sample thoroughly; the sample was allowed to stand at room temperature for 5 min for complete lysis, centrifuged at 12000 rpm at 4° C. for 5 min, the precipitate was discarded; 200 μL of chloroform was added, shaken vigorously for 15 s, placed at room temperature for 15 min, centrifuged at 4° C., 12000 rpm for 15 min, and the sample was divided into three layers; the upper layer of aqueous phase was taken and placed in a new 1.5 mL centrifuge tube, added with 0.5 mL of isopropanol, and turned upside down gently for well mixing, allowed to stand at room temperature for 5 min, centrifuged at 12,000 rpm at 4° C. for 10 min, the supernatant was discarded; 1 mL of 75% ethanol was added to the centrifuge tube, shaken gently, the precipitate was suspended, centrifuged at 8,000 g and 4° C. for 5 min, the supernatant was discarded; 1 mL of 75% ethanol was used to repeat washing once; the centrifuge tube was opened and placed on a clean bench, air-dried at room temperature for 5 min, added with 30 μL of RNase-free ddH₂O to dissolve RNA precipitate; 1 μL mixture was taken and loaded on Nanodrop 2000 to measure RNA concentration, and the sample that was not contaminated with proteins, phenols, inorganic salts, carbohydrates and other impurities and had a concentration greater than 125 ng/μL was taken for subsequent experiments.
(2) cDNA synthesis: M-MLV reverse transcription system (Promega, MSA) was used to synthesize cDNA from RNA, and its specific steps were as follows.

1 μg of RNA solution was added to a 0.2 mL PCR tube, supplemented with double distilled water (RNase-free) to make up the volume to 12 μL, added with 1 μL of random primers (if viral gene was to be cloned or detected, random primers should be used; if only eukaryotic gene was to be cloned, Oligo-dT primer could be used), mixed with pipette, centrifuged to precipitate it at the bottom of tube, then it was incubated at 70° C. for 60 min, subsequently incubated at 4° C. for 10 min; 5 μL of 5× MLV buffer, 5 μL of dNTP, 1 μL of reverse transcriptase, and 1 μL of RNase inhibitor were added to the above tube, mixed well with pipette, centrifuged to precipitate it to at the bottom of the tube, then it was incubated at 42° C. for 60 min, incubated at 75° C. for 15 min, and the reaction was stopped; the reverse transcription product was diluted according to the experimental requirements and stored in a −20° C. freezer.
(3) Primer design: The design of quantitative primers for each gene was shown in Table 4.

TABLE 4
Primer sequences for target genes

| Gene Name | Primer Sequence (5′-3′) |
|---|---|
| Q-OsFlot1 | F: GAACTACACCTTCGAGGTGC |
| | R: TGCGTCTTCTGGCCGAGGT |
| | F: TGTGGTGCTGTTCCAAGGTT |
| Q-OsIMP α4 | R: GGCTTCACTTGCACTTCAGC |
| RSV NP | F: GGAACAAATGCCAATGCTATC |
| | R: TGAGACATTTGGGAATAGCTGA |
| UBQ10 (rice | F: TGGTCAGTAATCAGCCAGTTTGG |
| reference gene) | |
| | R: GCACCACAAATACTTGACGAACAG |

(4) qPCR quantification: Talent qPCR PreMix (FP209-01) of Tiangen was used to perform qPCR reaction, and the reaction system was shown in Table 5. The reaction was carried out using a two-step PCR reaction procedure, and the reaction procedure was shown in Table 6. After attaching parafilm, centrifugation was performed to allow the reaction mixture to reach the bottom of tube. The reaction system was placed in a fluorescent quantitative PCR instrument (Thermo Pikoreal 96 Real-Time PCR System) to start the reaction.

TABLE 5
qPCR reaction system

| Component | System (20 μL) |
|---|---|
| Talent qPCR PreMix | 10 |
| Primer | 1 |
| cDNA template | 2 |
| RNase-Free ddH₂O | 7 |

TABLE 6
qPCR reaction procedure

| Stage | Cycle | Temperature | Time | | Content |
|---|---|---|---|---|---|
| Pre-denaturation | 1× | 95° C. | 15 | min | Pre-denaturation |
| PCR reaction | 40× | 95° C. | 30 | s | Denaturation |
| | | 60° C. | 30 | s | Annealing/extension |

### 2. Extraction of Rice Protein

The protein of rice was extracted using RIPA lysate kit (CW2333) of Kangwei Reagent. First, 5 ml of plant protein extraction reagent (containing 1% Protease inhibitor cocktail) was added to 1 g of tissue sample, mechanically homogenated and lysed; incubated on ice for 20-30 minutes; centrifuged at 13,400 g and 4° C. for 20 minutes; and supernatant was collected and stored at −20° C.

### 3. Western Experiment

After preparing 10% separating gel and 5% stacking gel; running was carried out at 80V for 30 min, after the sample entered the stacking gel, running was carried out at 120V for 1 h, and it was stopped when bromophenol blue run to the green line; 1 L of membrane transfer solution (200 ml of anhydrous methanol, 11.25 g of glycine, 3.025 g of Tris, and ultrapure water was added to reach 1 L); the membrane was activated in methanol for about 1 min; then, it was transferred into the membrane transfer solution: the transfer solution was poured into a tray, blackboard was placed face down, blackboard-sponge-filter paper-adhesive-membrane-filter paper-sponge-whiteboard were arranged in order, when each layer was added, a roller was used to pat out air bubbles once, after installation, it was placed in the membrane transfer tank, the blackboard was on the black side, and the whiteboard was on the red side; the membrane transfer tank was placed in an ice-water mixture, blocking was performed at 100V with 1 h:5% skimmed milk (Bio Yijie, BE6250) for 1 h, after shaking in a shaker at speed of 60 rpm/min: the milk was poured off, 5 ml of new milk was added, 1.5l of primary antibody (1:3000) was added and allowed to stand at room temperature for 2 h or overnight at 4° C., washing was carried out with PBST 3 times, each time for 5 min; 5 ml of milk was added, 1 μl of secondary antibody (1:5000) was added, reacted at room temperature for 1 h, washing was carried out with PBST for 3 times, each time for 5-15 min; ECL luminescent solution (Thermo, A38556) was used, A and B solutions were mixed at ratio of 1:1, and 400 μl thereof was added to each membrane, and imaging was carried out with a gel imaging device.

7. Rice Inoculation Experiment with RSV

When the rice seedlings grew to 2.5 leaf age, they are inoculated with virus, and each plant was inoculated with an average of 30 heads of virus-carrying SBPH, and they are clamped with micro-insect cages. The leaves eaten by the virus-carrying SBPH were the inoculated leaves, and the rest of rice leaves were the systemic leaves. After 2 days of vial inoculation, the virus-carrying SBPH and micro-insect cages were removed. The systematic leaves and the inoculated leaves were taken at 1d, 4d, and 7d, respectively, after the insects were removed, to perform quantitative detection of infection rate by qPCR. Each treatment had 8 biological replicates, the inoculated leaves were taken at 12 h and the systemic leaves were taken at 1d to perform plant immunofluorescence experiments to detect virus spread rate, and each treatment had 9-12 biological replicates. For the rice incidence rate, the inoculation was perform with two systems, in which each plant was inoculated with an average of 10 heads and 2 heads of virus-carrying SBPH, respectively, and they were clamped with micro-insect cages. After 2 days of inoculation, the virus-carrying SBPH and micro-insect cages were removed, the incidence rate was counted, and each treatment had 6 biological replicates.

### 8. Immunofluorescence Experiment of Rice Leaves

The inoculated rice leaves were fixed overnight at 4° C. with 4% paraformaldehyde (Collebo, SL18301); the tissue was transferred to a hard gelatin capsule (Electron Microscopy Sciences, 70102), and embedded with an embedding agent (Japanese Sakura, SAKMRA 4583), subjected to air evacuation, and quickly frozen at −20° C. for 5 minutes; it was then cut into 10 μm sections with a cryostat (Leica), placed on an adhesive slide, and air dried; the sections were placed in 10% goat serum (Beyotime, C0265) for 1 hour at room temperature to block non-specific binding sites; washed twice with PBST (containing 0.1% Tween 20); primary antibody was diluted with PBST (RSV NP mouse monoclonal antibody, 1:500), incubated overnight at 4° C., washed three times with PBST, and operation in dark started; secondary antibody (abberior, STORANGE-1002/1001) was diluted with PBST (1:1000), incubated at room temperature for 3 hours, and washed three times with PBST; each section was added with 20 μl of Aniline Blue Fluorochrome (Biosupplies, 100-1) at a concentration of 0.25 mg/mL, and 100 μl of anti-fluorescence quencher (BOSTER, AR1109), and plasmodesmata was stained at room temperature for 30 minutes; the slide was sealed with nail polish, and photoed with a laser confocal microscopy (Aniline Blue Fluorochrome, excitation wavelength: 390 nm, emission wavelength: 480 nm, STORANGE-1002); and each treatment had at least 6 biological replicates.

### Example 4: Studies on Rice Flotillin1 Mutant

### 1. Transcript Level and Protein Level

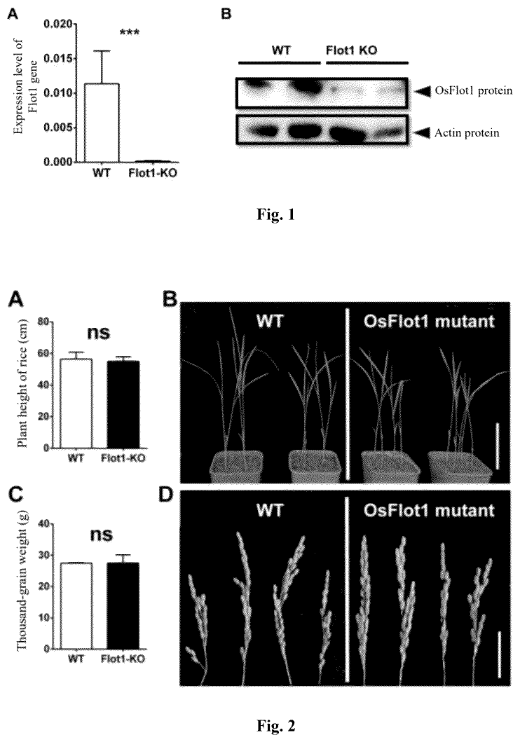

The results (FIG. 1) showed that at the transcriptional level, compared with wild-type rice WT, the expression level of Flotillin1 gene in Flotillin1 mutant rice was significantly reduced; at the protein level, compared with wild-type rice WT, the expression level of Flotillin1 protein in Flotillin1 mutant rice decreased significantly.

### 2. Effect of Knocking Out Flotillin1 Gene on Rice Plant Height, Thousand-Grain Weight and Germination Rate

Compared with WT, there was no significant difference in plant height and thousand-grain weight of rice in the Flotillin1 mutant (FIG. 2).

### 3. Effect of Knocking Out Flotillin1 Gene on Spread Rate of RSV in Rice

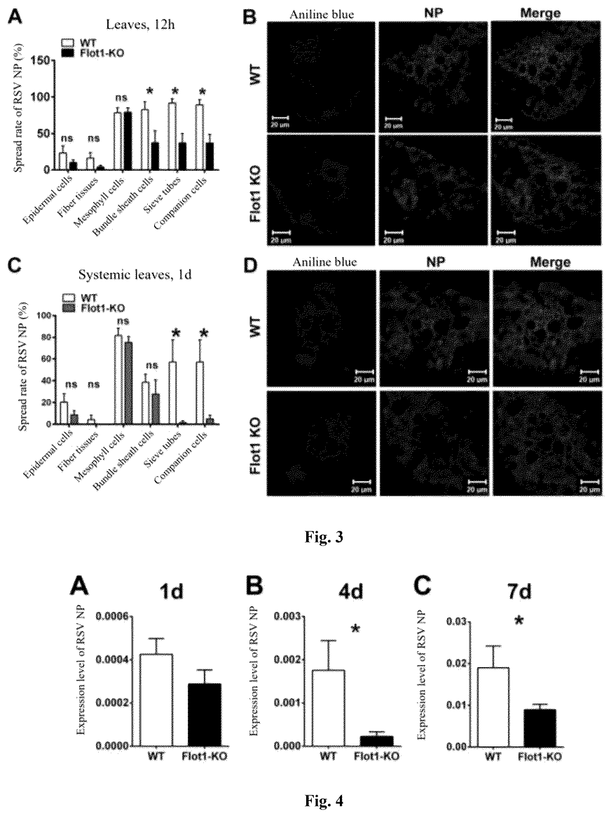

The effect of knocking out Flotillin1 on spread rate of RSV in rice was studied by plant immunofluorescence technology. The results (FIG. 3) showed that after 12 h of RSV inoculation in the inoculated leaves, compared with WT, in the Flotillin1 mutant, the spread rates of RSV in bundle sheath cells, sieve tubes and companion cells were significantly reduced by 55%, 36% and 34%, respectively; and after 1d of RSV infection in the systemic leaves, compared with WT, in the Flotillin1 mutant, the spread rates of RSV in sieve tubes and companion cells were significantly reduced by 64% and 57%, respectively.

### 4. Effect of Knocking Out Flotillin1 Gene on Expression Level of RSV NP in Rice

The effect of knocking out Flotillin1 gene on expression level of RSV NP in rice was studied by inoculating rice with highly RSV-loaded SBPH. The results (FIG. 4) showed that in the Flotillin1 mutant, compared with WT, there was no significant difference in the expression level of RSV NP when RSV infected leaves for 1 day, and when RSV infected leaves for 4 days and 7 days, RSV NP expression levels were significantly reduced by 87% and 53%, respectively.

### 5. Effect of Knocking Out Flotillin1 Gene on Rice Disease Incidence

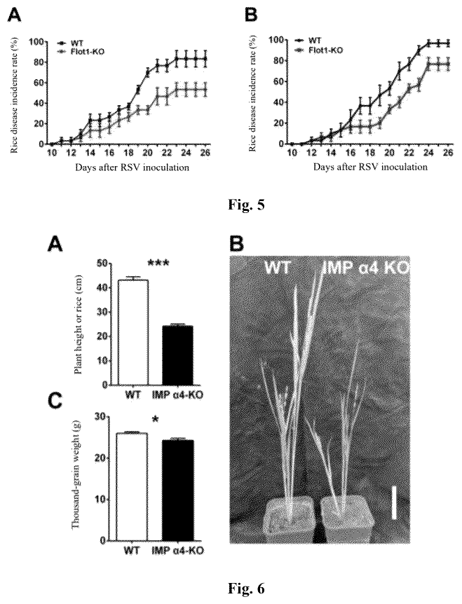

The effect of knocking out Flotillin1 gene on rice disease incidence was studied by inoculating rice with highly RSV-loaded SBPH. The results (FIG. 5) showed that in the Flotillin1 mutant, compared with WT, the incidence of RSV was significantly reduced by 20% when the population density was 10 heads, and the incidence of RSV was significantly reduced by 30% when the population density was 2 heads.

### Example 5: Studies on Importin α4 Mutant

### 1. Effect of Knockout of Importin α4 on Rice Plant Height and Thousand-Grain Weight

Compared with WT, in the Importin α4 mutant, the plant height and thousand-grain weight of rice decreased significantly (FIG. 6).

### 2. Effect of Knockout-Importin α4 on Expression Level of RSV NP in Rice

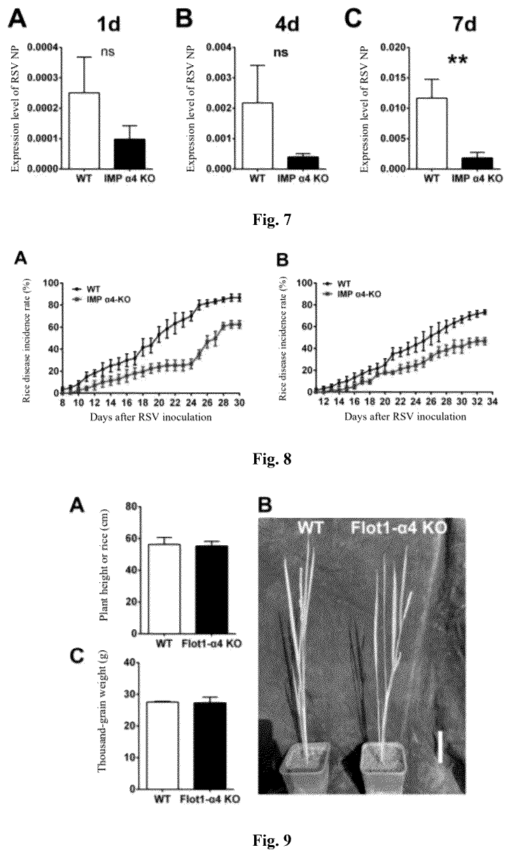

The effect of knocking out Flotillin1-Importin α4 on expression level of RSV NP in rice was studied by inoculating rice with highly RSV-loaded SBPH. The results (FIG. 7) showed that in the Importin α4 mutant, compared with WT, there was no significant difference in the expression of RSV NP when RSV infected the systemic leaves for 1d and 4d, while the expression level of RSV NP was significantly reduced by 84% when RSV infected the systemic leaves for 7d.

### 3. Effect of Knocking Out Importin α4 on Rice Disease Incidence

The effect of knocking out Importin α4 on rice disease incidence was studied by inoculating rice with highly RSV-loaded SBPH. The results (FIG. 8) showed that in the Importin α4 mutant, compared with WT, when the population density was 10 heads, the incidence of RSV was significantly reduced by 24% in the Importin α4 mutant, and when the population density was 2 head, the incidence of RSV was significantly reduced by 27%.

### Example 6: Study on Flotillin1-Importin α4 Mutant

### 1. Effect of Knocking Out Flotillin1-Importin α4 on Rice Plant Height and Thousand-Grain Weight

Compared with WT, there was no significant difference in plant height and thousand-grain weight of rice in the Flotillin1-Importin α4 mutant (FIG. 9).

### 2. Effect of Knocking Out Flotillin1-Importin α4 on Expression Level of RSV NP in Rice

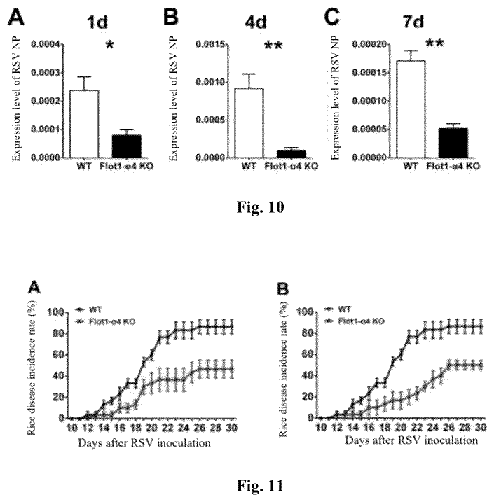

The effect of knocking out Flotillin1-Importin α4 on expression level of RSV NP in rice was studied by inoculating rice with highly RSV-loaded SBPH. The results (FIG. 10) showed that in the Flotillin1-Importin α4 mutant, compared with WT, the expression level of RSV NP in the Flotillin1-Importin α4 mutant was significantly reduced by 82%, 97%, and 70%, respectively, after RSV infected the systemic leaves for 1d, 4d and 7d.

### 3. Effect of Knocking Out Flotillin1-Importin α4 on Rice Disease Incidence

The effect of knocking out Flotillin1-Importin α4 on rice disease incidence was studied by inoculating rice with highly RSV-loaded SBPH. The results (FIG. 11) showed that in the Flotillin1-Importin α4 mutant, compared with the WT, when the population density was 10 heads, the incidence of RSV in the Flotillin1-Importin α4 mutant was significantly reduced by 37%, while when the population density was 2 heads, the incidence of RSV was significantly reduced by 40%.

Although the specific models for carrying out the present invention have been described in detail, those skilled in the art will understand that: according to all the teachings that have been disclosed, various modifications and changes can be made to the details, and these changes are all within the protection scope of the present invention. The full scope of the invention is given by the claims appended hereto and any equivalents thereof.

1. A method for obtaining a plant capable of resisting a pathogen, the method comprising: reducing or inhibiting the transcription or translation of Flotillin1 gene and Importin α4 gene in the plant, or reducing or inhibiting the expression level of proteins separately encoded by Flotillin1 gene and Importin α4 gene in the plant.

2.-12. (canceled)

13. The method according to claim 1, wherein the method has one or more characteristics selected from the group consisting of the following items:
(1) the plant is a gramineous plant;
(2) the pathogen is a virus;
(3) the Importin α4 gene is a rice Importin α4 gene; and,
(4) the Flotillin1 gene is a rice Flotillin1 gene.

14. The method according to claim 13, wherein the method has one or more characteristics selected from the group consisting of the following items:
(1) the plant is selected from the group consisting of wheat, barley, corn, rice, sorghum;
(2) the pathogen is a rice stripe virus;
(3) the protein encoded by the Importin α4 gene has an amino acid sequence as shown in SEQ ID NO: 3; and,
(4) the protein encoded by the Flotillin1 gene has an amino acid sequence as shown in SEQ ID NO: 1.

15. The method according to claim 1, wherein reducing or inhibiting the expression level of the plant Flotillin1 gene and Importin α4 gene is carried out by any one of the following methods: substitution, deletion or addition of one or several nucleotides, site-specific mutagenesis, ethyl methanesulfonate mutagenesis, directed induction of local mutation in genome, or gene editing.

16. The method according to claim 15, wherein substitution, deletion or addition 1, 2, or 3 nucleotides.

17. The method according to claim 1, wherein when there are several copies of Flotillin1 gene and Importin α4 gene in the plant cell, each copy of Flotillin1 gene and Importin α4 gene becomes defective.

18. The method according to claim 1, wherein the method is carried out through the following steps (a) to (f):
(a) constructing a vector containing sgRNA1 and sgRNA2, wherein the sgRNA1 is capable of targeting the Flotillin1 gene or fragment thereof, and the sgRNA2 is capable of targeting the Importin α4 gene or fragment thereof,
(b) transforming the vector into Agrobacterium;
(c) infecting plant cells with the Agrobacterium;
(d) optionally, selecting plant cells having defective Flotillin1 gene and Importin α4 gene;
(e) producing a plant from the plant cells of step (c) or (d);
(f) optionally, screening a plant with defective Flotillin 1 gene and Importin α4 gene to obtain the plant resisting to the pathogen.

19. The method according to claim 18, wherein the method has one or more characteristics selected from the group consisting of the following items:
(1) the sgRNA1 and sgRNA2 are constructed in the same vector or in different vectors;
(2) the Agrobacterium is EHA105 Agrobacterium;
(3) in step (f), RNA of the plant is extracted, the RNA is reversely transcribed into cDNA, primers are used to amplify nucleotide fragments targeted by the sgRNA1 and sgRNA2 in the cDNA for screening; alternatively, genomic DNA of the plant is extracted, and primers are used to amplify nucleotide fragments targeted by the sgRNA1 and sgRNA2 in the DNA for screening;
(4) the sgRNA1 has a nucleotide sequence as shown in SEQ ID NO: 5 or SEQ ID NO: 6; and,
(5) the sgRNA2 has a nucleotide sequence as shown in SEQ ID NO: 7 or SEQ ID NO: 8.

20. The method according to claim 1, wherein reducing or inhibiting the transcription or translation of Flotillin1 gene and Importin α4 gene in the plant, or reducing or inhibiting the expression level of proteins separately encoded by Flotillin1 gene and Importin α4 gene in the plant by using a nucleic acid molecule.

21. The method according to claim 20, wherein the method has one or more characteristics selected from the group consisting of the following items:
(1) the nucleic acid molecule is selected from antisense oligonucleotide, dsRNA, siRNA, shRNA, or sgRNA; and,
(2) the nucleic acid molecule comprises sgRNA1 and sgRNA2, the sgRNA1 is capable of targeting the Flotillin1 gene or fragment thereof, and the sgRNA2 is capable of targeting the Importin α4 gene or fragment thereof.

22. A kit, comprising any one or more of the following (1) to (6):
(1) a nucleic acid molecule that specifically inhibits the transcription or translation of Flotillin1 gene and Importin α4 gene, or specifically inhibits the expression level of proteins encoded by the Flotillin1 gene and the Importin α4 gene;
(2) a vector comprising the nucleic acid molecule;
(3) an Agrobacterium containing the vector;
(4) a medium and/or reagent for culturing plant cell or tissue;
(5) a reagent for extracting plant DNA or RNA; and,
(6) a primer set for amplifying a nucleotide fragment comprising the Flotillin1 gene and the Importin α4 gene.

23. The kit according to claim 22, wherein the kit has one or more characteristics selected from the group consisting of the following items:
(1) the nucleic acid molecule is selected from the group consisting of antisense oligonucleotides, dsRNA, siRNA, shRNA, or sgRNA; and,
(2) the nucleic acid molecule comprises sgRNA1 and sgRNA2, the sgRNA1 is capable of targeting the Flotillin1 gene or fragment thereof, and the sgRNA2 is capable of targeting the Importin α4 gene or fragment thereof.

24. The kit according to claim 22, wherein, the kit is used for obtaining a plant capable of resisting a pathogen, or is used for improving the ability of a plant to resist a pathogen.

25. The kit according to claim 22, the kit has one or more characteristics selected from the following items:
(1) the plant is a gramineous plant;
(2) the pathogen is a virus;
(3) the Importin α4 gene is a rice Importin α4 gene;
(4) the Flotillin1 gene is a rice Importin α4 gene;
(5) the vector is pYLCRISPR/Cas9P bi-H; and,
(6) the Agrobacterium is Agrobacterium EHA105.

26. The kit according to claim 25, the kit has one or more characteristics selected from the following items:
(1) the plant is selected from the group consisting of wheat, barley, corn, rice, sorghum;
(2) the pathogen is a rice stripe virus;
(3) preferably, the protein encoded by the Importin α4 gene has an amino acid sequence as shown in SEQ ID NO: 3; and,
(4) preferably, the protein encoded by the Flotillin1 gene has an amino acid sequence as shown in SEQ ID NO: 1.

27. The kit according to claim 23, wherein the kit has one or more characteristics selected from the group consisting of the following items:
(1) the sgRNA1 has a nucleotide sequence as shown in SEQ ID NO: 5 or SEQ ID NO: 6; and,
(2) the sgRNA2 has a nucleotide sequence as shown in SEQ ID NO: 7 or SEQ ID NO: 8.


SEQUENCE LISTING (20 sequences)

SEQ ID NO: 1 - protein, 485 aa, synthetic construct
MGFAYRIASASEYLAITGYGIADVKLAKKAWVAPGQRCTRFDISPVNYTFEVQAMSAEKLPFILPAVFTIGPRADDDDCLLRYAKLISPHDKLSHHVNELVKGVIEGETRVLAASMTMEEIFQGTKSFKQAVFENVQLELNQFGLIIYNANVKQLVDVAGHEYFSYLGQKTQQEAVNQAKVDVAEARMKGEVGAKERDGMTRQNAAKVDAETKVYTVKRQGEGAKEEARVKAEVKVFENEREAEVAEANADLAMKKAGWQRQAMVAEVEAAKAVAIREAELQVEVERTNASRQTEKLKAEHLSKAVVDYEMKVQEANWELYNRQKAAEALLYEQEKQAEARRASADAAFFARQREAEAELYAKQKEAEGLVAMGDAQSAYLSAMLGALGGSYAALRDYLMVSSGVYQEMARINADAIRGLEPKISVWSNGAGAGGEVGEGGGAMKEVAGVYKMLPPLLTTVHEQTGMLPPAWMGTLTGGAPSSTS

SEQ ID NO: 2 - protein, 1458 aa, synthetic construct
ATGGGGTTCGCGTACAGGATCGCGAGCGCGTCGGAGTACCTGGCGATCACCGGGTACGGCATCGCCGACGTGAAGCTGGCGAAGAAGGCGTGGGTGGCGCCGGGGCAGCGGTGCACCCGCTTCGATATCTCCCCGGTGAACTACACCTTCGAGGTGCAGGCCATGAGCGCCGAGAAGCTCCCCTTCATCCTCCCGGCCGTCTTCACCATCGGCCCCCGCGCCGACGACGACGACTGCCTCCTCCGCTACGCCAAGCTCATCTCCCCGCACGACAAGCTCTCCCACCACGTCAACGAGCTCGTCAAGGGCGTCATCGAGGGTGAGACCCGCGTGCTGGCCGCCTCCATGACCATGGAGGAGATCTTCCAGGGCACCAAGTCCTTCAAGCAGGCCGTCTTCGAGAACGTCCAGCTGGAGCTCAACCAGTTCGGCCTCATCATCTACAACGCCAACGTCAAGCAGCTCGTCGACGTCGCCGGCCACGAGTACTTCTCCTACCTCGGCCAGAAGACGCAGCAGGAGGCGGTGAACCAGGCGAAGGTGGACGTCGCGGAGGCGCGGATGAAGGGGGAGGTCGGCGCCAAGGAGAGGGACGGGATGACGCGGCAGAACGCCGCCAAGGTGGACGCCGAGACGAAGGTGTACACGGTGAAGCGGCAGGGCGAGGGGGCGAAGGAGGAGGCGAGGGTGAAGGCGGAGGTGAAGGTGTTCGAGAACGAGAGGGAGGCGGAGGTGGCCGAAGCGAACGCTGACCTGGCGATGAAGAAGGCCGGGTGGCAACGACAGGCGATGGTGGCTGAAGTGGAGGCCGCCAAGGCGGTCGCCATTCGTGAAGCCGAGCTGCAGGTGGAGGTGGAACGGACTAACGCCTCTAGGCAGACTGAGAAGCTCAAGGCCGAGCATCTCAGCAAGGCTGTCGTCGACTACGAGATGAAGGTGCAAGAAGCGAACTGGGAGCTGTACAACCGGCAGAAGGCGGCGGAGGCTCTGCTGTACGAGCAGGAGAAGCAGGCGGAGGCGCGGCGCGCGTCGGCGGACGCGGCCTTCTTCGCGCGGCAGCGCGAGGCCGAGGCGGAGCTCTACGCCAAGCAGAAGGAGGCCGAGGGCCTGGTGGCCATGGGCGACGCCCAGAGCGCCTACCTCTCCGCCATGCTCGGCGCGCTCGGCGGCAGCTACGCCGCGCTCCGGGACTACCTCATGGTCAGCTCCGGCGTGTACCAGGAGATGGCGCGCATCAACGCCGACGCCATCAGGGGGCTGGAGCCCAAGATCAGCGTGTGGAGCAACGGCGCCGGCGCCGGCGGCGAGGTCGGCGAAGGCGGTGGCGCGATGAAGGAGGTGGCCGGGGTGTACAAGATGCTGCCGCCGCTGCTGACGACGGTGCACGAGCAGACCGGGATGCTGCCGCCGGCGTGGATGGGCACTCTGACTGGCGGCGCCCCCTCGTCGACCAGTTGA

SEQ ID NO: 3 - protein, 456 aa, synthetic construct
MVEKVWSDDTTSQLEATIQFRRLLSDEKNPTVIKIIRADVLPRFSDFLSRHEHPQLQMEAAWVLTNIAASDYTLLVAECGAVPRLVELLESANANIRHQAIWALGNIAADVPTCREIVLDHGAVTPLLAQFREGMKVPVLRTATWALSNLCFGKLPAEVQVKPILDIISQLIHSVDEKILGDACWALCYICDGVSDGIQHVLDAGACPQLVNLLMHASANILLPVITVLARISSGDDAQVQVLVENDILNYLAPLLARNYPKSIKKQAYLIVSNISTGSKDNIQAVIDADVISPLIFLLKTSEKDIKEEAAWAISNAASGGSNDQIQYLVSRRCLEPLCNVLTYQDADLVYACLEGLQNILQAGAVGKQGQGSTVNPYAQFILECGGLDKLEDLQEVDNDAIYKLVMKLLEGYWDEEVSDDDPNLPTSNDSAETVETASEDAAQPTEPSASPNESE

SEQ ID NO: 4 - DNA, 1371 bp, synthetic construct
  source 1..1371: mol_type = other DNA
atggtggagaaggtctggtcagatgataccactagtcagttagaagccacaatccaattcaggagactcctttcagatgagaagaacccaaccgtgataaaaatcatcagagcagatgtcctgccaaggttttctgatttcctctcaagacatgagcatcctcagctacaaatggaggcggcatgggttcttaccaacatagctgcatctgactatacattgctagttgcagaatgtggtgctgttccaaggttggtcgagctcttagaatctgcaaatgctaatatcaggcatcaggctatctgggctcttggaaatatagctgcagacgtgcctacctgcagagagatcgtccttgatcatggtgctgtgacaccattacttgcccaatttagagaaggcatgaaagttccagttctgaggactgccacttgggcgttgtcaaacctttgttttggaaaattaccagctgaagtgcaagtgaagccaatactggatataatcagccagcttattcattcagtcgacgaaaagatactgggtgatgcatgctgggctctttgttatatatgtgacggtgtatctgacggaattcaacatgtgctagatgcgggtgcttgccctcaacttgtaaatcttttgatgcatgcatcagctaatatcctgcttcctgtcattacggtacttgcgagaatctcttctggagatgatgctcaagtgcaggtcctagtagaaaatgacattcttaattatttggccccgttgctagcacgaaattacccaaagagcatcaagaagcaagcttatctaattgtttctaatatctccactggcagcaaggataatattcaggcagtaattgatgcagatgttataagccccctcattttcctattaaagacctccgagaaggatatcaaggaggaagctgcttgggctatatcaaatgctgcgtctggtggttcaaatgatcaaattcaatatttggtgagtcgaagatgtttggagccactctgcaatgttctcacttaccaagatgctgacctagtatatgcgtgcctggaaggtcttcaaaacatacttcaggcgggtgcagtggggaagcaggggcagggttccacggtgaacccgtatgcacagtttattttagaatgtgggggtttggataaactggaagatctgcaggaagtcgacaatgatgccatttacaagttagtcatgaagttgctggagggttactgggacgaggaagtaagtgatgatgacccaaatttaccaacttcaaatgactctgcagagaccgtggagacggcatctgaagatgctgcgcagccaacagagccatcggccagcccaaatgaaagcgaatga

SEQ ID NO: 5 - DNA, 23 bp, synthetic construct
  source 1..23: mol_type = other DNA
gtgggagagcttgtcgtgcgggg

SEQ ID NO: 6 - DNA, 23 bp, synthetic construct
  source 1..23: mol_type = other DNA
ctcgaagacggcctgcttgaagg

SEQ ID NO: 7 - DNA, 23 bp, synthetic construct
  source 1..23: mol_type = other DNA
tcagagcagatgtcctgccaagg

SEQ ID NO: 8 - DNA, 23 bp, synthetic construct
  source 1..23: mol_type = other DNA
tctaagagctcgaccaaccttgg

SEQ ID NO: 9 - DNA, 20 bp, synthetic construct
  source 1..20: mol_type = other DNA
gaactacaccttcgaggtgc

SEQ ID NO: 10 - DNA, 19 bp, synthetic construct
  source 1..19: mol_type = other DNA
tgcgtcttctggccgaggt

SEQ ID NO: 11 - DNA, 20 bp, synthetic construct
  source 1..20: mol_type = other DNA
tgtggtgctgttccaaggtt

SEQ ID NO: 12 - DNA, 20 bp, synthetic construct
  source 1..20: mol_type = other DNA
ggcttcacttgcacttcagc

SEQ ID NO: 13 - DNA, 21 bp, synthetic construct
  source 1..21: mol_type = other DNA
ggaacaaatgccaatgctatc

SEQ ID NO: 14 - DNA, 22 bp, synthetic construct
  source 1..22: mol_type = other DNA
tgagacatttgggaatagctga

SEQ ID NO: 15 - DNA, 23 bp, synthetic construct
  source 1..23: mol_type = other DNA
tggtcagtaatcagccagtttgg

SEQ ID NO: 16 - DNA, 24 bp, synthetic construct
  source 1..24: mol_type = other DNA
gcaccacaaatacttgacgaacag

SEQ ID NO: 17 - DNA, 20 bp, synthetic construct
  source 1..20: mol_type = other DNA
gaactacaccttcgaggtgc

SEQ ID NO: 18 - DNA, 19 bp, synthetic construct
  source 1..19: mol_type = other DNA
tgcgtcttctggccgaggt

SEQ ID NO: 19 - DNA, 20 bp, synthetic construct
  source 1..20: mol_type = other DNA
tgtggtgctgttccaaggtt

SEQ ID NO: 20 - DNA, 20 bp, synthetic construct
  source 1..20: mol_type = other DNA
ggcttcacttgcacttcagc